(19) United States
(12) Patent Application Publication
(10) Pub. No.: US 2016/0038408 A1
(43) Pub. Date: Feb. 11, 2016

(54) METHODS FOR REDUCING ACUTE AXONAL INJURY

(71) Applicants: Board of Regents, the University of Texas System, Austin, TX (US); Baylor College of Medicine, Houston, TX (US)

(72) Inventors: Ping WU, League City, TX (US); Thomas A. Kent, Houston, TX (US)

(21) Appl. No.: 14/920,543

(22) Filed: Oct. 22, 2015

Related U.S. Application Data
(63) Continuation-in-part of application No. PCT/US2014/035182, filed on Apr. 23, 2014.
(60) Provisional application No. 61/815,072, filed on Apr. 23, 2013.

(51) Int. Cl. A61K 9/00; A61K 35/30; A61K 35/28; A61K 38/18

(52) U.S. Cl. CPC A61K 9/0043; A61K 38/185; A61K 35/30; A61K 35/28

(57) ABSTRACT

Provided herein are methods for treating an acute axonal injury by intranasally administering to a subject in need thereof an effective amount of a composition that includes a compound having the biological activity of inhibiting the effect of rapid stretch injury on neural stem-cell derived neurons. An example of such a compound is a Ret receptor ligand, such as a GDNF polypeptide. The compound is optionally associated with a delivery reagent. In one embodiment, the method further includes intranasally administering stem cells to the subject, such as neuronal stem cells or adipose-derived stem cells. Also provided herein are methods for decreasing impairment of long term potentiation of hippocampal synapses in a subject after an acute axonal injury.

### CROSS-REFERENCE TO RELATED APPLICATIONS

This application is a continuation in part of International Application No. PCT/US2014/035182, filed Apr. 23, 2014, published Oct. 30, 2014, as International Publication No. WO 2014/176360, which claims the benefit of U.S. Provisional Application Ser. No. 61/815,072, filed Apr. 23, 2013, each of which are incorporated by reference herein.

### GOVERNMENT FUNDING

The present invention was made with government support under PT074693P6, awarded by the Department of Defense. The government has certain rights in this invention.

### BACKGROUND

Acute axonal injury (AAI) is a traumatic injury to the brain caused by an external force. AAI often results from violence, transportation accidents, construction, and sports, and populations at risk of AAI include youth involved in certain sports, athletes, and military personnel. AAI may cause long-term disabilities in cognition, emotion, sensory, and/or motor functions. It is a risk factor in Alzheimer's disease, and repetitive mild AAI may lead to chronic traumatic encephalopathy (CTE). Despite considerable progress, clinical treatment is still limited to supportive care.

Traumatic axonal injury (TAI) or diffuse axonal injury is a key pathological feature of AAI. Damage in a TAI occurs over a more widespread area, and is associated with all levels of AAI, from mild to severe. White matter damage from the forces causing brain injuries is usually present as the result of serial molecular, physiological, and structural changes, including axolemmal disruption, intracellular calcium accumulation, loss of microtubules, neurofilament compaction, mitochondrial damage, calpain-mediated proteolysis, axonal swelling, and secondary axotomy (Wang et al., 2012, J. Neurotrauma, 29:295-312, Maxwell et al., 1997, J. Neurotrauma, 14:419-440, Povlishock, 1992, Brain Pathol., 2:1-12). TAI contributes to both mortality and morbidity in AAI patients, and is central to the impact of AAI on life quality and performance. Because TAI is a process persisting hours to days and even years after initial injury, amelioration of TAI could result in a significant functional improvement in AAI patients.

Glial cell-derived neurotrophic factor (GDNF) is a small highly conserved neurotrophic protein of 134 amino acids (monomer is approximately 14.7 kDa) that is present as a dimer. GDNF promotes the survival of many types of neurons, and also promotes neurite regrowth. GDNF has been shown to have potential as a treatment for Parkinson's disease. Monkeys with an induced form of Parkinson's disease showed less trembling when treated with the drug, and neuronal fibres grew in part of the human brain exposed to the drug. However, the use of GDNF in the treatment of neurological conditions requires delivery of the GDNF through the blood-brain barrier.

The blood-brain barrier (BBB) is a boundary that separates circulating blood from the brain extracellular fluid present in the brain and central nervous system. Endothelial cells that line the vessels in the brain and central nervous system restrict the diffusion of microscopic objects (e.g., bacteria) and large or hydrophilic molecules into the brain and central nervous system, while allowing the diffusion of small hydrophobic molecules (such as O₂, CO₂, and hormones). The BBB presents a significant barrier to the delivery of therapeutic agents to the brain. When used to treat neurological conditions such as Parkinson's disease, GDNF has been administered to patients by invasive intracerebral infusions (Kastin et al., 2003, Neurosci. Lett., 340:239-241). This route of administration poses significant risks including life-threatening infections, intracerebral hemorrhages, embolic strokes, and even death.

The potential of GDNF for AAI repair has been examined in animals through various methods including GDNF released from grafted stem cells (Gao, et al., 2006, Exp. Neurol., 201:281-292; Cheng et al., 2008, Cell Res., 18:215-217; Wang et al., 2012, J. Neurotrauma., 29:295-312), intracerebroventricular injection of GDNF (Kim et al., 2001, J Neurosurg., 95:674-679), or intracerebral injection of a viral vector containing the GDNF gene (Minnich et al., 2010, Restor Neurol Neurosci., 28:293-309). However, those methods are invasive, and not suitable for the majority of AAI patients who suffer from mild to moderate AAI. Intranasal delivery of GDNF has also been tested in animal models for Parkinson's disease, but resulted in the insufficient delivery of GDNF to the structurally normal brain in Parkinson's disease (Migliore M M, 2008, “Intranasal delivery of GDNF for the treatment of Parkinson's disease,” Doctoral Dissertation, Pharmaceutical Science Dissertations. Paper 1).

### SUMMARY OF THE APPLICATION

A significant obstacle to the use of therapeutics for AAI and other neurodegenerative conditions is the presence of the blood-brain barrier (BBB). It is believed that greater than 98% of all small drug molecules, and approximately 100% of large drug molecules are excluded from the brain by the BBB (Pardridge, 2005, NeuroRx 2:3-14). Small drug molecules tend to have a molecular mass of less than 500-Da, and be highly lipophilic such that they form less than 8-10 hydrogen bonds with water in solution before they cross the BBB in therapeutically sufficient levels (Pardridge, 2005, NeuroRx, 2:3-14).

There are six predominant transport mechanisms that naturally exist in the BBB: paracellular, transcellular, facilitated transport, receptor mediated endocytosis, adsorptive endocytosis, and carrier mediated efflux transport (Neuwelt, 2004, Neurosurgery, 54:131-140). The four main types of techniques investigated for delivery of drugs and therapeutic proteins across the BBB and into the brain are: BBB disruption, bypassing the BBB, using chimeric translocating proteins, and using delivery reagents to deliver drugs to the brain.

BBB disruption attempts to increase paracellular transport by interfering with tight junction formation and/or integrity. For instance, hyperosmolar solutions may cause a disruption of the BBB by causing endothelial cells to dehydrate and shrink, thereby loosening tight junctions and increasing intercellular space. However, BBB disruption carries risks associated with possible increased transfer of toxic agents, including pathogens, into the brain.

Bypassing the BBB is another method that has been employed in order to deliver drugs to the brain. One pathway is through the nasal mucosa. Compounds that are taken up by the nasal mucosa and transported by transcellular mechanisms into the brain tend to be lipophilic, low molecular weight molecules, and small polar molecules and peptides tend to be much less amenable to effective intranasal administration (see, for example, Illum, 2003, J Control Release, 87:187-198.). Compounds may also be transported through the nasal mucosa and into the brain by paracellular mechanisms, and the molecular weight cut off for compounds that can be transported in this way has been reported to be up to 26,500 kDa.

Chimeric peptide technology has also been used to improve BBB permeability. This approach takes advantage of receptor-mediated endocytosis mechanisms to breach the BBB. For instance, compounds have been conjugated with an anti-transferrin antibody, insulin, or the HIV TAT polypeptide to allow transport of the compounds across the BBB (Bradbury et al., 2000, The Blood-Brain Barrier and Drug Delivery to the CNS. New York, N.Y.: Marcel Dekker, Inc; Torchilin et al., 2001, Proc Natl Acad Sci USA, 98:8786-8791; Drin et al., 2003, J. Biol. Chem., 278:31192-31201).

Delivery reagents, such as vesicles and particles, have also been used.

The inventors have made the unexpected and surprising observation that intranasal delivery of a large protein, ovalbumin, is efficient with a wide distribution of ovalbumin into the injured brain. Furthermore, they provide strong evidence to support the proposition that acute intranasal delivery of GDNF reduces axonal injury immediately after AAI in rats, which may have long-term effects to prevent chronic consequences from AAI, e.g. reducing the risk of Alzheimer's Disease and/or chronic traumatic encephalopathy that occurs after repetitive mild AAI such as occurring in boxers, football players, military personnel.

Provided herein are methods for treating an acute axonal injury. The methods include administering to a subject in need thereof an effective amount of a composition that includes one or more compounds that reduce axonal injury after AAI. In one embodiment the compound is one having the biological activity of inhibiting the effect of rapid stretch injury on neural stem-cell derived neurons. In one embodiment, such a compound is a Ret receptor ligand, such as glial cell line-derived neurotrophic factor (GDNF), Neurturin, Artemin, or Persephin. In one embodiment the compound is a GDNF mimic. A combination of compounds described herein may also be administered. In one embodiment, the administration is intranasal. In one embodiment, the GDNF polypeptide is r-metHuGDNF. The polypeptide may be a fusion polypeptide.

Also provided herein is a method for improving long term potentiation in a subject after an acute axonal injury. The method includes intranasally administering to a subject in need thereof an effective amount of a composition that includes a compound, such as a Ret receptor ligand or a GDNF mimic, and a pharmaceutically acceptable carrier, wherein the administration results in decreasing impairment of long term potentiation of hippocampal synapses.

In one embodiment, a method described herein may further include intranasally administering an effective amount of stem cells to the subject. In one embodiment, the stem cells may be neuronal stem cells or adipose-derived stem cells. The stem cells may be administered before, during, and/or at the same time as the composition that includes a compound, such as a Ret receptor ligand or a GDNF mimic. In one embodiment, prior to administration the adipose-derived stem cells are cultured in conditions suitable for differentiation of the adipose-derived stem cells into neuronal cells. In one embodiment, the subject receiving the stem cells has a moderate or severe acute axonal injury.

In one embodiment, the compound may be associated with a delivery reagent, such as a liposome, micelle, polymersome, or nanparticle. In one embodiment, the compound is not associated with a delivery reagent. In one embodiment, at least one dose of a compound is administered within 1, 6, 12, 24, 48, or 72 hours after an acute axonal injury. In one embodiment, at least one dose is administered within 6 hours after an acute axonal injury. In one embodiment, at least one dose is administered within 1 hour of an acute axonal injury. In one embodiment, at least one dose of stem cells is administered within 1, 6, 12, 24, 48, or 72 hours after an acute axonal injury. In one embodiment, the duration of treatment is equal to or less than 1 day, 3 days, 7 days, 14 days, or 30 days. In one embodiment, the compound is administered in a dose amount of between 5 mg and 15 mg per day. In one embodiment, the compound is administered in a total amount of between 30 uL to 300 uL. In one embodiment, the acute axonal injury is a diffuse axonal injury or a focal axonal injury. In one embodiment, the acute axonal injury is a mild to severe acute axonal injury. In one embodiment, the acute axonal injury is a repetitive traumatic brain injury. In one embodiment, the acute axonal injury is spinal cord injury. In one embodiment, the composition is formulated as an intranasal spray, an intranasal aerosol, or a nasal drop.

Also provided herein is a pharmaceutical formulation for intranasal administration that includes at least 1 microgram per microliter (ug/ul) of a compound, such as a Ret receptor ligand or a GDNF mimic, wherein the compound is not associated with a delivery reagent.

As used herein, the term “polypeptide” refers broadly to a polymer of two or more amino acids joined together by peptide bonds. The term “polypeptide” also includes molecules which contain more than one polypeptide joined by a disulfide bond, or complexes of polypeptides that are joined together, covalently or noncovalently, as multimers (e.g., dimers, tetramers). Thus, the terms peptide, oligopeptide, enzyme, and protein are all included within the definition of polypeptide and these terms are used interchangeably. It should be understood that these terms do not connote a specific length of a polymer of amino acids, nor are they intended to imply or distinguish whether the polypeptide is produced using recombinant techniques, chemical or enzymatic synthesis, or is naturally occurring.

As used herein, “identity” refers to sequence similarity between two polypeptides or two polynucleotides. The sequence similarity between two polypeptides is determined by aligning the residues of the two polypeptides (e.g., a candidate amino acid sequence and a reference amino acid sequence, such as SEQ ID NO:1) to optimize the number of identical amino acids along the lengths of their sequences; gaps in either or both sequences are permitted in making the alignment in order to optimize the number of shared amino acids, although the amino acids in each sequence must nonetheless remain in their proper order. The sequence similarity is typically at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity. Sequence similarity may be determined, for example, using sequence techniques such as the BESTFIT algorithm in the GCG package (Madison Wis.), or the Blastp program of the BLAST 2 search algorithm, as described by Tatusova, et al. (FEMS Microbiol Lett 1999, 174:247-250), and available through the World Wide Web, for instance at the internet site maintained by the National Center for Biotechnology Information, National Institutes of Health. Preferably, sequence similarity between two amino acid sequences is determined using the Blastp program of the BLAST 2 search algorithm. Preferably, the default values for all BLAST 2 search parameters are used, including matrix=BLOSUM62; open gap penalty=11, extension gap penalty=1, gap x_dropoff=50, expect=10, wordsize=3, and optionally, filter on. In the comparison of two amino acid sequences using the BLAST search algorithm, structural similarity is referred to as “identities.”

Conditions that “allow” an event to occur or conditions that are “suitable” for an event to occur, such as an enzymatic reaction, or “suitable” conditions are conditions that do not prevent such events from occurring. Thus, these conditions permit, enhance, facilitate, and/or are conducive to the event. Such conditions, known in the art and described herein, may depend upon, for example, the enzyme being used.

As used herein, a polypeptide “fragment” includes any polypeptide which retains at least some of the activity of the corresponding native polypeptide. Examples of fragments of polypeptides described herein include, but are not limited to, proteolytic fragments and deletion fragments.

As used herein, a “delivery reagent” refers to a reagent that can aid in the transfer of a compound, such as a polypeptide, across the nasal mucosa and into the brain. Examples of delivery reagents include, but are not limited to, vesicles (including liposomes, polymersomes), particles (including nanoparticles), and micelles. Other delivery reagents include mannitol, phosphatidylserine, olive oil, and chitosan (Hanson et al., 2012, Drug Delivery, 19:149-54, Feng et al., 2012, Int. J. Pharmaceutics, 423:226-234). A delivery reagent is associated with a compound through one or more non-covalent interactions. For instance, a compound may be physically enclosed in a delivery vehicle, such as a compound present as a cargo in the interior compartment of a liposome. In another aspect, a compound may be bound to a delivery reagent by an ionic bond, a hydrogen bond, a Van der Waals force, or a combination thereof.

As used herein, “pharmaceutically acceptable,” means that the compositions or components thereof so described are suitable for use in contact with human mucosa without undue toxicity, incompatibility, instability, allergic response, and the like.

The term “and/or” means one or all of the listed elements or a combination of any two or more of the listed elements.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims.

Unless otherwise specified, “a,” “an,” “the,” and “at least one” are used interchangeably and mean one or more than one.

Also herein, the recitations of numerical ranges by endpoints include all numbers subsumed within that range (e.g., 1 to 5 includes 1, 1.5, 2, 2.75, 3, 3.80, 4, 5, etc.).

For any method disclosed herein that includes discrete steps, the steps may be conducted in any feasible order. And, as appropriate, any combination of two or more steps may be conducted simultaneously.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Neuronal death in pig hippocampi after TBI. (A) Low magnification (40×) typical adolescent male pig sham control shows both CA1 (#1) and CA3 (#2) hippocampal regions. (B) Higher magnification (100×) typical adolescent male sham control CA3 hippocampus. (C) Typical male CA3 hippocampus (100×) at 4 hr after a moderate fluid percussion injury. (D) Summary data for mean number of necrotic neurons in CA1 and CA3 hippocampus of adolescent male pigs under conditions of sham control and TBI, n=5. *p<0.05 compared to corresponding sham control value.

FIG. 2. Level of GDNF in rats and pigs after intranasal GDNF delivery. (A-C) Intranasal GDNF (T+G) increases the level of GDNF in rat CSF, cortex and hippocampus when compared to the vehicle control (T+V)(n>3). The ELISA data are average pg GDNF normalized by total mg protein; means±SEM, *p<0.05, one way ANOVA with post tests. (D-F) GDNF delivery also increases the GDNF level in pig CSF and brain regions (n=1). Sham, control without injury; T, trauma; V, vehicle control; G, GDNF.

FIG. 3. Intranasal GDNF treatment reduces pathological changes after moderate fluid percussive injury (FPI). (A-D) GDNF reduces lesion size (arrow) 3 days after injury. (E-K) GDNF reduces axonal injury. (E) FPI causes an abnormal accumulation of APP in the external capsule white matter (arrows) and hippocampal fimbria (arrowhead). The arrowhead-pointed region is enlarged in (H) and quantified in (K). (F) The low dose (8 μg/day) of GDNF results in a dramatic blockade of APP accumulation in the fimbria region (arrowhead, enlarged in I), and a significant reduction in the external capsule damage (arrows in F). (G) The high dose (24 μg/day) of GDNF nearly abolishes APP accumulation in both the external capsule (arrows) and the fimbria (arrowhead in G is enlarged in J). (L-O) GDNF reduces α-smooth muscle actin (α-SMA, a stress fiber component). (L) FPI induces the expression of α-SMA in the epicenter of injury (arrow) and the opposite side of the brain along the injury axis (i.e., the left lower corner, arrowhead). The low dose GDNF significantly reduces α-SMA elevation (M), and the high dose near completely abolishes α-SMA (N). (P-S) GDNF reduces tau oligomer accumulation. FPI induces abnormal accumulation of tau oligomer ipsilaterally (arrow in P). The low dose slightly reduces tau accumulation (Q), and the high dose dramatically diminishes the elevated tau oligomers (R). D, K, O and S are quantitative analyses of the averaged area (pixels²) or intensities (pixels per fixed area), 3 sections per rat brain spanning 150 μm along the A-P axis.

FIG. 4. Rescue of impaired long term potentiation (LTP) with intranasal GDNF. (A) Hippocampal LTP was induced using the taburst stimulation (TBS) in brain slices from normal rats (open circles), from rats with TBI treated with vehicle (solid circles), and from rats with TBI treated with GDNF (solid circles with X). Excitatory postsynaptic currents (EPSCs) were evoked at the fimbira-CA3 synapse. (B-D) individual examples:traces show averages of 8-10 EPSCs recorded in CA3 pyramidal cells before and 60 min after TBS. (E) GDNF-mediated improvement in short-term spatial learning and memory 12-days post injury (TBI+G). Morris water maze working memory tests were performed to determine the time to reach platform. Each rat had 4 pairs of swimming, two Trials per pair and a 4-min interval between pairs. Average of the 2^{nd} trials from 4 pairs per rat are plotted. *p<0.05 compared to Sham and TBI+G, n=5-6. (F) Novel object recognition test to monitor recognition memory at 30 days post injury. Animals were subjected to habituation, familiarization of objects, and then test with a novel object. Exploratory preference is calculated by dividing the time spent for exploring novel object by the total time. *p<0.05 compared to Sham and TBI+GDNF, n=5-6. One-way ANOVA with Tukey's test.

FIG. 5. Stem cell-secreted GDNF reverse traumatic injury-induced expression of α-SMA in rat hippocampal tissues. Western blot analyses were performed on protein extracts from rat hippocampi 15 days post-injury. Sham, control without injury; T+H, fluid percussion TBI plus hemorrhagic shock (blood withdrawal to reach MAP of 40 mm Hg for 40 min); T+H+V, TBI plus hemorrhage followed by vehicle injection at 1 day post injury; T+H+C, TBI plus hemorrhage followed by human neural stem cell (hNSC) transplantation; T+H+C+IgG, TBI plus hemorrhage followed by hNSC grafting and intraparenchymal infusion of control antibody; T+H+C+αGDNF, TBI plus hemorrhage followed by hNSC grafting and infusion of GDNF neutralizing antibody. Values are expressed as means±SEM. ***p<0.001, one way ANOVA plus Tukey's tests.

FIG. 6. Changes of GDNF receptors and downstream signaling molecules in rat hippocampi. (A-B) Increased expressions of GDNF family receptor α1 (GRFα1) and co-receptor RET were detected in the injured rat hippocampi 2 weeks post TBI. (B-F) Compared to animals that received neural stem cell transplantation after TBI, those treated additionally with a GDNF neutralizing antibody showed decreases in phosphorylated RET (pRET), phospho-Akt and phospho-ERK1/2, while an increase in phosphor-ROCK2. Sham, control without injury; TBI, 2-atm lateral fluid percussion injury; T+C, transplanted with primed human neural stem cells one day post TBI; T+C, received cell transplant and a 7-day of anti-GDNF infusion. (A-B) Data represent means±SEM, n=6. *p<0.05, **p<0.01, one way ANOVA plus Tukey's tests.

FIG. 7. Cell death and signaling changes in neural stem cell-derived neurons/astrocytes after stretch injury and GDNF treatment. (A) About one third of cells died/dying at 1.5 hr after stretch injury (SI) detected by phase contrast and staining with Propidium iodide or Fluoro Jade C. Scale bars, 20 μm. (B) Western blot analyses of phosphorylated RET, Akt, ERK1/2 and ROCK2 after stretch injury (SI) or SI plus GDNF treatment (SI+G) at 1.5 hr post injury. Human neural stem cells were seeded to BioFlex plates and differentiated into neurons/astrocytes for 10 days, which were then subjected to 60 psi (regulator pressure) stretch injury under the Cell Injury Controller II. Thirty minutes later, cells were treated with 15 ng/ml GDNF or vehicle; and then subjected to morphological testing and Western blot analyses at 1.5 hrs post-injury. Sham, control without injury; SI, 60 psi stretch injury with vehicle; SI+G, GDNF 30 min post stretch injury.

FIG. 8. Changes of signaling molecules in pig hippocampi after intranasal GDNF delivery. (A) Compared with traumatic brain injury alone (T), GDNF treatment (T+G) increases phosphorylation of Akt (pAkt) in the pig hippocampus. (B) GDNF also increases phosphorylated ERK1/2 (pERK1/2). Methods: Western blot analyses were performed on protein extracts from adolescent pig hippocampi 30 minutes after intranasal GDNF administration or 1.5 hours after traumatic brain injury. T, 2-atm lateral fluid percussion injury; T+G, 1 mG of GDNF being intranasally delivered into a pig 1 hour post injury; N=1

### DETAILED DESCRIPTION OF ILLUSTRATIVE EMBODIMENTS

The present invention includes methods for using compounds that promote neurite regrowth and/or neuron survival. In one embodiment, a compound is a polypeptide. In one embodiment, examples of polypeptides include members of the Ret receptor ligand family of neurotrophic factors, such as glial cell line-derived neurotrophic factor (GDNF), Neurturin, Artemin, and Persephin. Other polypeptides include those that mediate downstream signaling by GDNF. In another embodiment, a compound is a GDNF mimic. Examples of GDNF mimics include small molecules and proteins, such as compounds that activate Dok-4 and/or Rap1GAP, compounds that block RhoA/ROCK signals, compounds that block PTEN signals, compounds that activate PI3K/Akt, compounds that activate cAMP, compounds that activate ERK1/2, and compounds that activate Rac1.

A compound useful herein has biological activity. Whether a compound has biological activity may be determined by in vitro assays. Preferably, an in vitro assay is carried out by determining whether a test compound inhibits the effect of rapid stretch injury on neural stem-cell derived neurons (Wang et al., 2012, J. Neurotrauma, 29:295-312). As described by Wang et al., rapid stretch injury of neural stem-cell derived neurons causes significantly shortened lengths of axons and dendrites, increases the expression of both amyloid precursor protein and α-smooth muscle actin, and induces actin aggregation. A test compound that decreases or prevents any one or all of those effects indicates the test compound has biological activity.

An example of a GDNF is depicted at SEQ ID NO:1. The sequence depicted at SEQ ID NO:1 is available from the Genbank database as amino acids 78-211 of accession number CAG46721.1, where a methionine has been added to the amino terminal end of the processed polypeptide. Other examples of GDNF polypeptides useful in the methods described herein include those having sequence similarity with the amino acid sequence of SEQ ID NO:1. A GDNF polypeptide having sequence similarity with the amino acid sequence of SEQ ID NO:1 has GDNF activity. A GDNF polypeptide may be isolated from a cell, such as a human cell, or may be produced using recombinant techniques, or chemically or enzymatically synthesized using routine methods.
SEQ ID NO:1: MSPDKQMAVLPRRERNRQAAAANPENSRGKGRRGQRGKNRGCVLTAIH LNVTDLGLGYETKEELIFRYCSGSCDAAETTYDKILKNLSRNRRLVSDKV GQACCRPIAFDDDLSFLDDNLVYHILRKHSAKRCGCI

The amino acid sequence of a GDNF polypeptide having sequence similarity to SEQ ID NO:1 may include conservative substitutions of amino acids present in SEQ ID NO:1. A conservative substitution is typically the substitution of one amino acid for another that is a member of the same class. For example, it is well known in the art of protein biochemistry that an amino acid belonging to a grouping of amino acids having a particular size or characteristic (such as charge, hydrophobicity, and/or hydrophilicity) may generally be substituted for another amino acid without substantially altering the secondary and/or tertiary structure of a polypeptide. For the purposes of this invention, conservative amino acid substitutions are defined to result from exchange of amino acids residues from within one of the following classes of residues: Class I: Gly, Ala, Val, Leu, and Ile (representing aliphatic side chains); Class II: Gly, Ala, Val, Leu, Ile, Ser, and Thr (representing aliphatic and aliphatic hydroxyl side chains); Class III: Tyr, Ser, and Thr (representing hydroxyl side chains); Class IV: Cys and Met (representing sulfur-containing side chains); Class V: Glu, Asp, Asn and Gln (carboxyl or amide group containing side chains); Class VI: His, Arg and Lys (representing basic side chains); Class VII: Gly, Ala, Pro, Trp, Tyr, Ile, Val, Leu, Phe and Met (representing hydrophobic side chains); Class VIII: Phe, Trp, and Tyr (representing aromatic side chains); and Class IX: Asn and Gln (representing amide side chains). The classes are not limited to naturally occurring amino acids, but also include artificial amino acids, such as beta or gamma amino acids and those containing non-natural side chains, and/or other similar monomers such as hydroxyacids. A GDNF polypeptide having sequence similarity to SEQ ID NO:1 may include at least 1, at least 2, at least 3, at least 4, at least 5, at least 6, at least 7, at least 8, at least 9, or at least 10 conservative substitutions.

A GDNF polypeptide is a small highly conserved neurotrophic protein of 135 amino acids (approximately 14.7 kDa). It has GDNF activity as a homodimer. Thus, an assay to determine if a polypeptide has GDNF activity is accomplished using a dimer. A GDNF polypeptide monomer includes two long fingers connected by loops, and a helical region at the opposite end (Eigenbrot and Gerber, 1997, Nature Struct. Biol., 4:435-438). Two monomers associate in a tail-to-head configuration, with the two helices flanking a cysteine-knot motif at the center of the structure (Ekethall et al., 1999, EMBO J., 18:5901-5910). The amino acids forming the fingers, helical region, and cysteine-knot are conserved. Also conserved is the pattern of cysteine residues, as well as the net positive charge formed across the middle of the dimer and negatively charged residues clustered at the end of the monomer forming a patch of negative electrostatic potential (Ekethall et al., 1999, EMBO J., 18:5901-5910).

The other members of the Ret receptor ligand family, Neurturin, Artemin, and Persephin, are known to the skilled person in the art. Amino acid sequences of examples of each of these polypeptides are readily available, and methods for determining whether a polypeptide has Neurturin, Artemin, or Persephin, activity are known in the art and routinely used. Also included herein are polypeptides having Neurturin, Artemin, or Persephin activity and having sequence similarity with a Neurturin, Artemin, or Persephin polypeptide. Compounds that activate Dok-4 and Rap1GAP, compounds that block RhoA/ROCK signals, compounds that block PTEN signals, compounds that activate PI3K/Akt, compounds that activate cAMP, compounds that activate ERK1/2, and compounds that activate Rac1 are also known to the skilled person and are readily available.

In one embodiment, a polypeptide described herein, such as a Ret receptor ligand, may be a fragment. For example, in one embodiment a GDNF polypeptide useful herein may be a fragment of SEQ ID NO:1 or a polypeptide having sequence similarity to SEQ ID NO:1. In one embodiment, a GDNF polypeptide fragment may be missing one or more amino acids from the amino terminal end, for instance, at least 1, at least 2, at least 3, at least 4, at least 5, at least 6, at least 7, at least 8, at least 9, at least 10, at least 15, at least 20, at least 25, at least 30, at least 35, or at least 40 amino acid residues compared to a full length GDNF polypeptide. In one embodiment, a GDNF polypeptide fragment may be missing one or more amino acids from one or more regions between the amino acids that make up the fingers and the helix.

In one embodiment, a polypeptide described herein, such as a Ret receptor ligand or a fragment thereof may include additional amino acids, provided the additional amino acids do not prevent the resulting amino acid sequence from having biological activity. For instance, a GDNF polypeptide having SEQ ID NO:1 or sequence similarity to SEQ ID NO:1 may include 1, 2, 3, 4, 5 6, 7, 8, 9, 10, or more amino acids at the amino terminal end, 1, 2, 3, 4, 5 6, 7, 8, 9, 10, or more amino acids at the carboxy terminal terminal end, or a combination thereof. In one embodiment, a GDNF polypeptide may include a methionine residue at the amino terminal end (e.g., r-metHuGDNF, also available under the trade designation Liatermin (Amgen)). In one embodiment, the additional amino acids may impart a specific function, such as the ability to breach the BBB. A Ret receptor ligand that includes additional amino acids that aid in moving the Ret receptor ligand across the BBB is referred to herein as a “fusion polypeptide.” Such amino acids sequences include, but are not limited to, those that aid in using receptor-mediated endocytosis mechanisms to move a Ret receptor polypeptide across the BBB. Examples of such amino acid sequence include, but are not limited to, anti-transferrin antibody, insulin, and the HIV TAT polypeptide.

A compound, such as a Ret receptor ligand, for instance, a GDNF polypeptide, useful in the methods described herein may be present in a composition. In one embodiment, a composition includes a pharmaceutically acceptable carrier. Additional active compounds can also be incorporated into a composition. As used herein “pharmaceutically acceptable carrier” includes, but is not limited to, saline, solvents, dispersion media, coatings, antibacterial and antifungal agents, isotonic and absorption delaying agents, and the like, compatible with pharmaceutical administration. Some examples of suitable carriers include water, salt solutions, alcohols, polyethylene glycols, polyhydroxyethoxylated castor oil, peanut oil, olive oil, gelatin, lactose, terra alba, sucrose, dextrin, magnesium carbonate, sugar, cyclodextrin, amylose, magnesium stearate, talc, gelatin, agar, pectin, acacia, stearic acid or lower alkyl ethers of cellulose, silicic acid, fatty acids, fatty acid amines, fatty acid monoglycerides and diglycerides, pentaerythritol fatty acid esters, polyoxyethylene, hydroxymethylcellulose and polyvinylpyrrolidone.

In one embodiment, the route of administration of a composition described herein is intranasal to the nasal epithelium. Intranasal delivery can be accomplished by formulating the compound, such as GDNF, as an intranasal spray, an intranasal aerosol, or a nasal drop. In another embodiment, routes of administration include parenteral (e.g., intravenous, intradermal, subcutaneous, intraperitoneal, intramuscular), enteral (e.g., oral), and topical (e.g., epicutaneous, transmucosal) administration.

Formulations can be mixed with auxiliary agents which do not deleteriously react with the active agent, e.g., a compound described herein, for instance Ret receptor ligand, such as a GDNF polypeptide. Such additives can include wetting agents, emulsifying and suspending agents, salt for influencing osmotic pressure, buffers and/or coloring substances preserving agents, sweetening agents or flavoring agents. The compositions can also be sterilized if desired.

If a liquid carrier is used, the preparation can be in the form of a liquid such as an aqueous liquid suspension or solution. Acceptable solvents or vehicles include sterilized water, Ringer's solution, or an isotonic aqueous saline solution.

The active agent may be provided as a powder suitable for reconstitution with an appropriate solution as described herein. Examples of these include, but are not limited to, freeze dried, rotary dried or spray dried powders, amorphous powders, granules, precipitates, or particulates. The composition can optionally contain stabilizers, pH modifiers, surfactants, bioavailability modifiers and combinations of these. A unit dosage form can be in individual containers or in multi-dose containers.

Compositions may include, for example, a delivery reagent, such as micelles, liposomes, polymersomes, nanoparticles, or microparticle, or can be administered in an extended release form to provide a prolonged storage and/or delivery effect, e.g., using biodegradable polymers, e.g., polylactide-polyglycolide. Examples of other biodegradable polymers include poly(orthoesters) and poly(anhydrides). In certain embodiments, a compound described herein, such as a Ret receptor ligand, including a GDNF polypeptide, is not associated with a delivery reagent.

A composition containing a compound described herein can be formulated to provide quick, sustained, controlled, or delayed release, or any combination thereof, of the active agent after administration to the individual by employing procedures well known in the art. In one embodiment, the active agent is in an isotonic or hypotonic solution. In one embodiment, for an active agent that is not water soluble, a lipid based delivery vehicle may be employed, e.g., a microemulsion or liposomes.

Mucociliary clearance mechanisms can rapidly remove compounds delivered to the nasal epithelium, reducing contact with the nasal epithelium and delivery into the brain after intranasal administration. Mucoadhesive agents, e.g., sodium hyaluronate, chitosan, acrylic acid derivatives, lectin, and low methylated pectin, surface-engineered nanoparticles, efflux transporter inhibitors, and vasoconstrictors, may be used to reduce clearance, to prolong the residence time of the formulation at the delivery site, and to increase transport from the nasal epithelium to the brain.

The active agent may be effective over a wide dosage range. For example, in the treatment of adult humans, dosages from 5 mg to 15 mg per day may be used. In choosing a regimen for individual it can frequently be necessary to begin with a higher dosage and when the condition is under control to reduce the dosage. The exact dosage will depend upon the activity of the compound and the form in which administered, as well as, for instance, the extent of injury suffered by the individual, the body weight of the individual to be treated, and the experience of the physician in charge.

Dosage forms suitable for nasal administration may include an appropriate amount of the active agent mixed with a pharmaceutically acceptable carrier to result in the administration of an effective amount to a subject. In one embodiment, the compound, for instance a Ret receptor ligand such as a GDNF polypeptide is 10 mg/mL to 30 mg/mL. In one embodiment, a volume of 30 uL to 300 uL is administered per human nostril.

Dosage forms can be administered daily, or more than once a day, such as two or three times daily. Alternatively dosage forms can be administered less frequently than daily, such as every other day, or weekly, if found to be advisable by a prescribing physician.

Nasal delivery devices, such as sprays, atomized sprays, nose droppers or needle-less syringes, may be employed to target the agent to different regions of the nasal cavity. Examples of devices include OptiMist (Optinose), ViaNase (Kurve Technology), Go-Pump (Braun), Versidose (Mystic Pharmaceuticals), Accuspray (3M), and MAD Nasal (LMA).

Toxicity and therapeutic efficacy of the active compounds can be determined by standard pharmaceutical procedures in cell cultures or experimental animals, e.g., for determining the ED₅₀ (the dose therapeutically effective in 50% of the population). The data obtained from cell culture assays and/or animal studies can be used in formulating a range of dosage for use in humans. The dosage of such active compounds lies preferably within a range of concentrations that include the ED₅₀ with little or no toxicity. The dosage may vary within this range depending upon the dosage form employed and the route of administration used. For an active compound used in the methods of the invention, it may be possible to estimate the therapeutically effective dose initially from cell culture assays. A dose may be formulated in animal models to achieve a concentration range that includes the IC₅₀ (i.e., the concentration of the test compound which achieves a half-maximal inhibition of signs and/or symptoms) as determined in cell culture. Such information can be used to more accurately determine useful doses in humans.

Also provided are methods for using the compounds disclosed herein, such as Ret receptor ligands, including a GDNF polypeptide. In one embodiment, a method includes administering to a subject in need thereof an effective amount of a compound. In one embodiment, the subject may have sustained an acute axonal injury (AAI), such as a traumatic axonal injury (TAI) or diffuse axonal injury. The AAI may be mild (e.g., concussion), moderate, or severe, and may be a repetitive AAI. Without intending to be limited by theory, methods disclosed herein may result in protection of neurons, reduction of axonal damage, promotion of axon regrowth, and/or prevention chronic consequences following AAI. In one embodiment, the administration is under conditions suitable for movement of the compound from the nasal epithelium to the brain.

As used herein, an “effective amount” relates to a sufficient amount of a compound, such as a polypeptide, to provide the desired effect. For instance, in one embodiment an “effective amount” is an amount effective to protect neurons, reduce axonal damage, promote axon regrowth, and/or prevent chronic consequences following AAI. In one embodiment, an “effective amount” is an amount sufficient to improve or alleviate clinical signs, such as motor function, sensory function, mood behaviors, or a combination thereof, following AAI. In one embodiment, an “effective amount” is an amount sufficient to inhibit the effect of rapid stretch injury on neural stem-cell derived neurons,

In one embodiment, a method of the present invention includes treating certain conditions. In one embodiment, a method includes treating a condition in a subject, where a subject in need thereof is administered an effective amount of a composition that includes a compound described herein, such as a Ret receptor ligand, including a GDNF polypeptide. The subject may be a mammal, such as a member of the family Muridae (a murine animal such as rat or mouse), a primate, (e.g., monkey, human), a dog, a sheep, a guinea pig, or a horse. As used herein, the term “condition” refers to any deviation from or interruption of the normal structure or function of a part of the central nervous system, such as the brain, of a subject that is manifested by a characteristic symptom or clinical sign. Conditions include, but are not limited to, AAI, TAI, diffuse axonal injury, chronic traumatic encephalopathy (CTE), and cognitive impairment.

As used herein, the term “symptom” refers to subjective evidence of disease or condition experienced by the patient. As used herein, the term “clinical sign,” or simply “sign,” refers to objective evidence of a disease or condition present in a subject. Symptoms and/or signs associated with diseases or conditions referred to herein and the evaluation of such signs are routine and known in the art, and may include magnetic resonance imaging and/or diffusion tensor imaging. Examples of signs of a condition may include, but are not limited to, altered motor function, altered sensory function, altered mood behaviors, e.g., eye or verbal response, and/or impaired memory, e.g., impaired long-term potentiation. In one embodiment, a method described herein is useful in treating impairment of long-term potentiation of CA3-CA1 synapses in the hippocampus. A condition may be assessed by any accepted neurological or ICU scale, such as the Glasgow Coma Scale (GCS), Acute Physiology and Chronic Health Evaluation II (APACHE II), Simplified Acute Physiology Score (SAPS II), and Sequential Organ Failure Assessment (SOFA). For example, AAI can be classified on the Glasgow scale as mild (11-15), moderate (7-10), or severe (3-6). Whether a subject has a condition, and whether a subject is responding to treatment, may be determined by evaluation of signs associated with the condition.

Treatment of a condition can be prophylactic or, alternatively, can be initiated after the development of a disease or condition. Treatment that is prophylactic, for instance, initiated before a subject manifests signs of a condition, is referred to herein as treatment of a subject that is “at risk” of developing a condition. An example of a subject that is at risk of developing a condition is a person taking part in an activity that is likely to result in AAI. Treatment can be performed before or after the occurrence of the conditions described herein. Treatment initiated after the development of a condition may result in decreasing the severity of the signs of the condition, or completely removing the signs. An “effective amount” may be an amount effective to alleviate one or more symptoms and/or signs of the condition. In one embodiment, an effective amount is an amount that is sufficient to effect a reduction in a symptom and/or sign associated with a disease or condition. A reduction in a symptom and/or a sign is, for instance, at least 10%, at least 20%, at least 30%, at least 40%, at least 50%, at least 60%, at least 70%, at least 80%, at least 90%, or at least 100% in a measured sign as compared to a control, a non-treated subject, or the subject prior to administration of the compound.

A composition described herein may be administered as soon as possible to a subject that has been exposed to conditions that may result in AAI, TAI, diffuse axonal injury, CTE, and/or cognitive impairment, for instance, having received a trauma to the head. The trauma may be, for example, a diffuse axonal injury, focal axonal injury, mild to severe traumatic brain injury, repetitive traumatic brain injury, concussion, or spinal cord injury. In one embodiment, the composition may be delivered within 10 minutes, within 30 minutes, within 1 hour, within 3 hours, within 6 hours, within 12 hours, within 1 day, within 2 days, within 3 days, within 4 days, within 5 days, within 6 days, within 7 days, within 14 days, within 30 days, etc. In one embodiment, the composition may be delivered as 1 dose per day, 2 doses per day, 3 doses per day, 4 doses per day, or 5 doses per day.

In one embodiment, a method described herein further includes administering to the subject a composition that includes a stem cell. In one embodiment, the number of cells administered may be at least 1, at least 100, at least 1,000, at least 10,000, or at least 100,000 cells. In one embodiment, the number of cells may be no greater than 10,000,000, no greater than 1,000,000, or no greater than 10,000 cells. Stem cells have been shown improve cognitive function after traumatic injury to the brain (Gao, et al., 2006, Exp. Neurol., 201:281-292; Wang et al., 2012, J. Neurotrauma., 29:295-312), and it is expected that stem cells can aid in treating a subject having one of the conditions described herein. Examples of suitable stem cells include neural stem cells and adipose-derived stem cells. Methods for obtaining neural stem cells (Svendsen, C. N, et al., 1998 J Neurosci Methods 85:141-152; Wu P 2002 Nat Neurosci., 5(12):1271-1278) and adipose-derived stem cells (Yu et al., 2011, Methods Mol. Biol., 702:17-27) are known to the skilled person. In one embodiment, adipose-derived stem cells are differentiated into neuronal or neuronal precursor cells before administration to a subject. Methods for culturing adipose-derived stem cells under conditions to cause differentiation into neuronal or neuronal precursor cells are known to the skilled person (Cardozo et al., 2012, Gene, 511(2):427-436; Yang et al., 2014, PLoS One, 9(1):e86334). The route of administration of the stem cells is intranasal to the nasal epithelium. The stem cells may be administered at the same time as a composition that includes a compound, such as a Ret receptor ligand or a GDNF mimic, after the composition is administered, before the composition is administered, or a combination thereof. In one embodiment, a composition that includes stem cells may be delivered within 10 minutes, within 30 minutes, within 1 hour, within 3 hours, within 6 hours, within 12 hours, within 1 day, within 2 days, within 3 days, within 4 days, within 5 days, within 6 days, within 7 days, within 14 days, within 30 days, etc. In one embodiment, a composition that includes stem cells may be delivered as 1 dose per day, 2 doses per day, 3 doses per day, 4 doses per day, or 5 doses per day. In one embodiment, the subject has AAI that is mild, moderate, or severe. In one embodiment, the AAI is moderate or severe.

In certain embodiments, because the trauma is acute, the duration of treatment is limited to less than or equal to 1, 3, 7, 14, or 30 days.

The present invention is illustrated by the following examples. It is to be understood that the particular examples, materials, amounts, and procedures are to be interpreted broadly in accordance with the scope and spirit of the invention as set forth herein.

### Example 1

### Use of Pigs as a Model for Traumatic Brain Injury

Rodents and primates traditionally have been the preferred animal species in neuroscience. However, use of pigs within neuroscience has increased dramatically over the last 20 years (se, for instance, Armstead et al., 2009, J Cereb Blood Flow Metab 29 (3):524-533, Armstead et al., 2010, J Neurotrauma 27 (2):391-402). The emergence of pig experimental models reflects the considerable resemblance of pigs to human anatomy and physiology. Rodents have a lissencephalic brain containing more grey than white matter. In contrast, pigs have a gyrencephalic brain that contains substantial white matter similar to humans. White matter is more sensitive to traumatic damage than grey matter. The pig is widely available to commercial production, presenting a considerable advantage over primates for ethical and economic reasons.

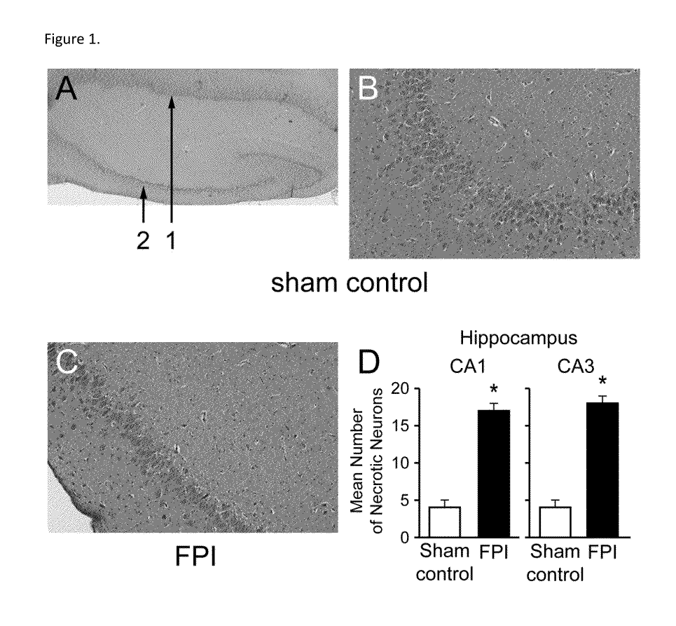

Our preliminary data show neuronal death in the CA3 region of pig hippocampi 4 hours after a moderate fluid percussion injury (FPI) (FIG. 1). Neuronal pathology scoring (blinded) was assessed by counting damaged neurons/1.2 mm² in the CA1 and CA3 regions: mild (1-5), moderate (6-15), and severe (>15). These data indicate that this level of FPI caused severe damage in the adolescent pig

### Example 2

### Intranasal Administration of GDNF

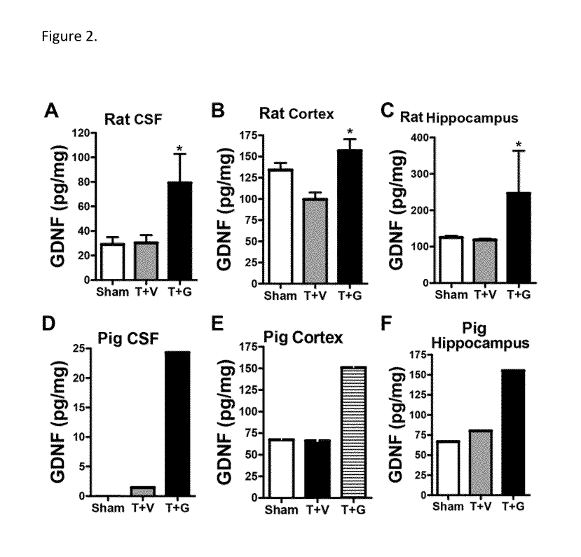

Intranasal delivery of GDNF into structurally normal rodent brains has been proven insufficient (Migliore, 2008, Intranasal delivery of GDNF for the treatment of Parkinson's disease. Pharmaceutical Science Dissertations Paper 1), probably due to the large size and positively charged nature of GDNF. Traumatic brain injury (TBI), on the other hand, is often associated with various degrees of anosmia, which is may be accompanied with a disruption of the tight junction between olfactory epithelial cells (Hasegawa et al., 1986, Arch Otorhinolaryngol 243(2):112-116, Kern et al., 2000, Laryngoscope 110(12):2106-2109). The resultant leaky nasal mucosa may allow large molecules readily access to CSF. We carried out trial experiments in both adolescent rats and pigs by intranasally giving one dose of GDNF over a 1 hour period of time at clinically relevant window of time (1 to 3 hour after a moderate fluid percussion injury, a model of TBI), 50 μg/100 μl/each for rats and 1.1 mg/2.2 ml/each for pigs based on the brain weight ratio between rat and pig. The preliminary data clearly show that intranasal GDNF induced significant increases of GDNF in CSF and critical brain regions such as hippocampus and cortex from both rats (58% to 1.6-fold increases, n=3-5) and pigs (94% to 15.1-fold increases, n=1) at 30 min after a single dose of GDNF administration (FIG. 2).

### Example 3

We show for the first time that an acute intranasal administration of glial cell line-derived neurotrophic factor (GDNF) can dramatically reduce brain damage after head injury in rats. Specifically, intranasal GDNF delivery within 6 hours reduces traumatic axonal injury, the common feature occurring in all levels of traumatic brain injury (TBI) ranging from mild to severe and central to the impact of TBI on the quality of life in patients.

### Methods and Materials

Rats were subjected to moderate fluid percussion injury (FPI at 2 atm), and received the first dose of GDNF treatment via intranasal administration at six hours post injury. Two more doses of GDNF were then delivered in two subsequent days, i.e. 30 and 54 hours following injury. On day 4 (or 75 hours after injury), anesthetized animals received intracardiac perfusion of 4% paraformaldehyde. Brain tissues were collected and subjected to histological analyses to evaluate the level of Traumatic axonal injury (TAI) and the TAI-related molecular changes.

Moderate FPI was induced as described previously (Gao, et al., 2006, Exp. Neurol., 201:281-292; Wang et al., 2012, J. Neurotrauma., 29:295-312). The animals were adult male Sprague Dawley rats, approximately 10-11 weeks old, and 325 to 350 grams. All animal surgeries were conducted according to NIH Guide for the Care and Use of Laboratory Animals, and proved by the Institutional Animal Care and Use Committee.

The administration of GDNF in PBS (without any carrier or delivery reagent) was accomplished as described by Migliore (“Intranasal delivery of GDNF for the treatment of Parkinson's disease.” (2008) Pharmaceutical Science Dissertations. Paper 1) with some modifications. Isoflurane-anesthetized rats were placed in a supine position with their noses at an upright 90° angle. The GDNF used was obtained from Cell Guidance Systems LLC (Carlsbad, Calif.). A cohort of 3 rats received intranasal delivery of phosphorylated saline (PBS) as controls. Two rats received GDNF at a dosage of either 24 or 72 μg/day. GDNF or PBS in a volume of 2 μl was dropped by P10 pipette into one nostril at a time, alternating the nostrils every 2 minutes until each nostril received a total of 10 μl. Animals were then kept in supine position for 45-60 minutes.

### Results

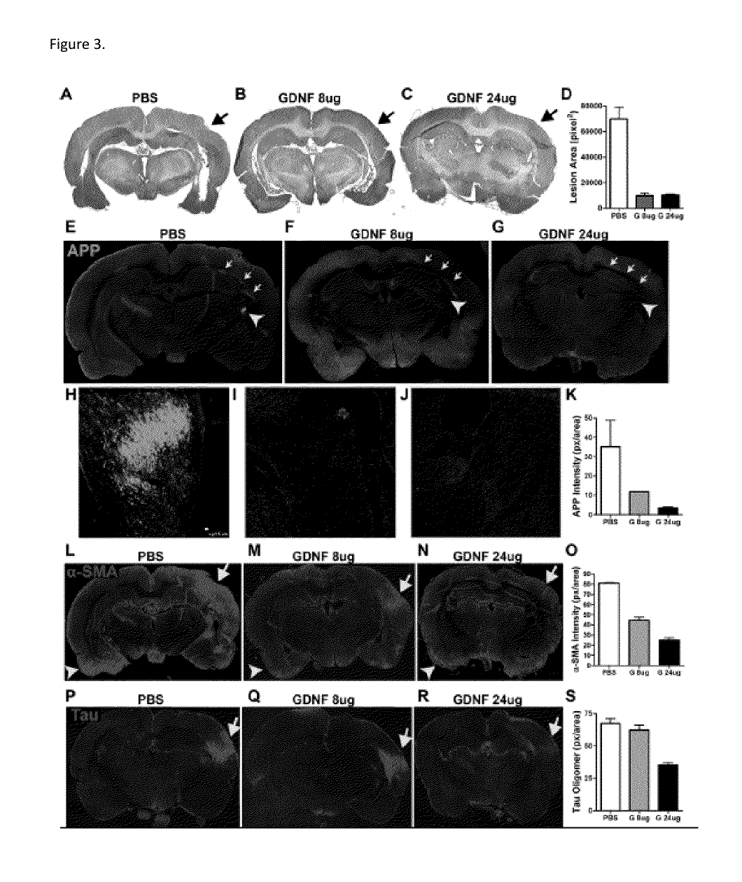

Compared to the PBS controls, intranasal GDNF administration, just 3 doses within 54 hours post moderate FPI, efficiently blocked or reduced TAI, which was identified by the accumulation of beta-amyloid precursor protein (βAPP). βAPP is an axonal injury marker. Furthermore, acute GDNF treatment reduced the injury-induced expression of alpha smooth muscle actin (α-SMA), and elevation of tau in a dose-dependent manner. Indeed, we found, to our surprise, that intranasal GDNF delivery (starting at 6 hours post injury, one dose per day for 3 days), significantly and dose-dependently reduced the lesion size (FIG. 3A-D), accumulation of APP (a marker for axonal injury, FIG. 3E-K), and abnormal α-SMA expression (FIG. 3L-O). Particularly striking is that intranasal GDNF (high dose) significantly decreased the injury-induced oligomeric tau detected by a tau oligomer antibody (FIG. 3P-S) (Hawkins et al., 2013, J. Biol. Chem., 288(23):17042-50). GDNF also reduced tau phosphorylation. Thus, our preliminary data demonstrated the feasibility of intranasal GDNF delivery in rats, which strongly support the effectiveness of intranasal GDNF as a novel therapy for TBI.

### Conclusion

Our data show that acute GDNF intranasal administration effectively reduced TAI after moderate TBI in rats.

### Example 4

To get an idea of how far intranasally delivered GDNF spread in rat brains, we applied an indirect tracing technique using ovalbumin, since the endogenous GDNF interferes with the tracking of intranasal GDNF. We also compared the intranasal route with the intracerebroventricular route in two different TBI models: mild fluid percussive injury (FPI) and mild blast injury in rats.

### Methods and Materials

Rats received either mild FPI (1 atm) or mild blast injury to the right brains. Six hours later, a fluorophore (Alex596)-conjugated ovalbumin (a protein of 45 kDa) was delivered either intranasally using the same technique as for GDNF above, or through intracerebroventricular infusion. For intranasal delivery, each rat received one dose of 50 ug Alex596-ovalbumin and sacrificed the next day. For the intracerebroventricular route, animals received 0.7 μg/day through an Alzet osmotic pump for 3 days and then sacrificed for brain analyses.

### Results

Intranasal delivery of ovalbumin resulted in widespread fluorescent deposits throughout the whole brain, whereas intracerebroventricular administration yielded a rather restricted distribution mainly to the delivery side. More intense deposition was shown in the mild blast injury (mBINT) brains than those with mild FPI. Fluorescent deposits appeared to be either diffuse punctations or concentrated within the cells. Compared to the faint and smooth appearance of fluoresce in sham-injured brains, stronger and coarse granular deposits were detected in mBINT and mild FPI brain regions, including cortex, hippocampus, thalamus and hypothalamus.

Conclusion

These data indicate that the intranasal route is an effective way to administer ovalbumin into a wide range of the rat brain following mild TBI. Given that the size of ovalbumin (45 kDa) is bigger than that of GDNF (30 kDa), it is conceivable that GDNF delivered intranasally also distributes widely in the injured rat brains, and thus executes its effects against TAI.

### Example 5

The GDNF's protective effect against Traumatic axonal injury (TAI) has also been confirmed in an in vitro cell injury model.

### Methods and Materials

Primarily cultured human fetal brain-derived neural stem cells were differentiated into neurons previously described (Wu et al., 2002, Nat Neurosci., 5(12):1271-1278). Human neurons were then subjected to a rapid stretch injury (30 psi) as described (Wang et al., 2012, J. Neurotrauma., 29:295-312). Thirty minutes after injury, cells were either left untreated, or treated with 15 ng/ml GDNF (R&D Systems) with or without a specific neutralizing antibody for GDNF (R&D Systems). Four days later, cells were fixed by 4% paraformaldehyde and analyzed by immunostaining with antibodies for APP or α-SMA.

### Results

Rapid stretch injury induced upregulation and accumulation of 13-APP and α-SMA in human neurons. GDNF treatments efficiently reduced 13-APP and α-SMA, which was blocked by GDNF specific antibodies.

### Conclusion

The in vitro injury model mimics the rat in vivo TBI model, and confirms that GDNF is a potent reagent to block traumatic axonal injury.

### Example 6

### Intranasal GDNF Rescues the Electrophysiological Function of Neurons

TBI results in neural disconnection and/or loss of synaptic transmission. Several previous studies have shown impaired long-term potentiation (LTP) at CA3-CA1 synapses when recorded in the injured hippocampal slices from juvenile to adult rats. However, none examined the fimbria pathway, which contains important afferent and efferent fibers connecting the hippocampus with other brain regions and frequently damaged following TBI.

### Materials and Methods

Rats received a moderate FPI (2 atm), and then two doses of GDNF intranasal delivery, each with 24 μg at 1 and then 6 hour post injury (n=2). Brain slices were collected 1 day post injury and subjected to a procedure similar to the procedure described by Czeh et al. (Czeh et al., 1990, Brain Res., 518(1-2):279-282). The stimulate probe was located in the fimbria, and recording probe was in the CA3 region.

### Results

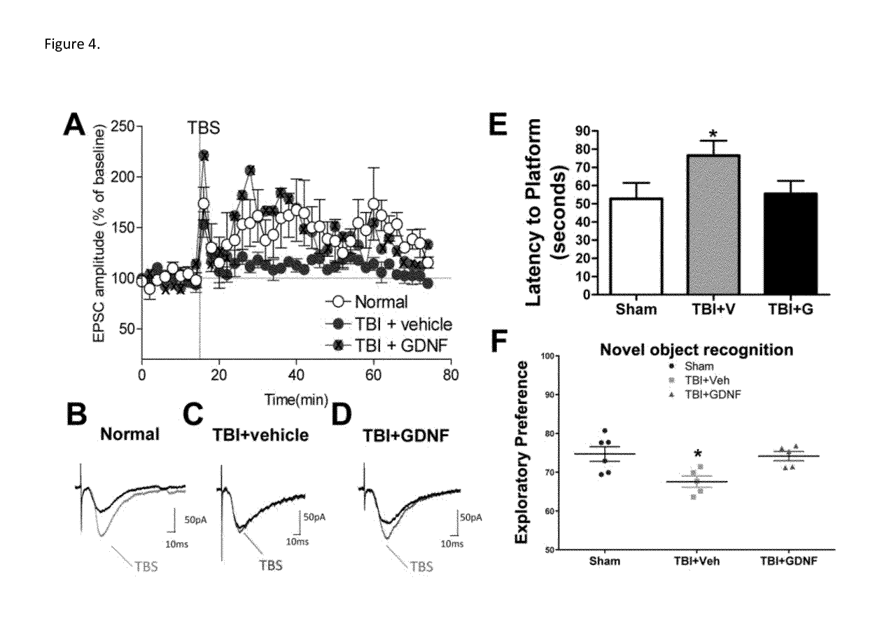

Intranasal administration of GDNF, two doses starting at 1 hour post injury, improved the electrophysiological function of the hippocampal neurons as shown by preserving both Excitatory postsynaptic current (EPSC) and LTP properties of the CA3 pyramidal neurons after injury (FIG. 4A-D)), and the short-term spatial memory measured by Morris water maze test (FIG. 4E). A trend of improved recognition memory was also evident by the novel object recognition test (FIG. 4F). For electrophysiological study, brain slices were collected 24 hr after TBI. Hippocampal long-term potentiation (LTP) was induced using the theta burst stimulation (TBS) and excitatory postsynaptic currents (EPSCs) were evoked at the fimbira-CA3 synapse. GDNF rescued TBI-induced LTP impairments (TBI+GDNF, FIG. 4A-D). GDNF also reduced latency to reach a platform as assessed by the Morris Water Maze test at 12 days post TBI (FIG. 4E), and enhanced the New Object Recognition preference (FIG. 4F).

### Conclusion

Acute intransal administration of GDNF efficiently protected the electrophysiological function of the hippocampal neruons, which is critical for learning and memory.

### Example 7

### Blocking GDNF by a Neutralizing Antibody Reduces GDNF Neuroprotective Effects

To determine the effect of cell transplantation on traumatic axonal injury, we first confirmed the occurrence of traumatic brain injury by detecting abnormal accumulation of amyloid precursor protein (APP) in injured rat brains, and then discovered that transplantation of human neural stem cells completely blocked the APP accumulation (Wang, et al., 2012, J Neurotrauma 29:295-312). More interestingly, we revealed for the first time that a smooth muscle protein, alpha smooth muscle actin (α-SMA), was significantly elevated in the neurons of the injured Hippocampal regions four days after TBI, and cell grafting diminished the injury-induced expression of α-SMA. We hypothesize that α-SMA is cell response to injury, but also blocks axon regrowth and neuronal reconnection.

### Materials and Methods

Rats were subjected to a 2.0-atm parasagittal fluid percussion TBI, followed by hemorrhagic hypotension through withdrawing blood from the jugular vein to keep the blood pressure down to 40 mm Hg for 40 minutes. The Sham group went through the whole surgical preparation, including craniotomy and jugular vein cannulation, but without fluid percussion injury. One day later, TBI rats were divided into five groups: receiving nothing, or intrahippocampal injection of vehicle, 10⁵ human neural stem cells, cells plus GDNF neutralizing antibody, or cells plus control IgG.

### Results

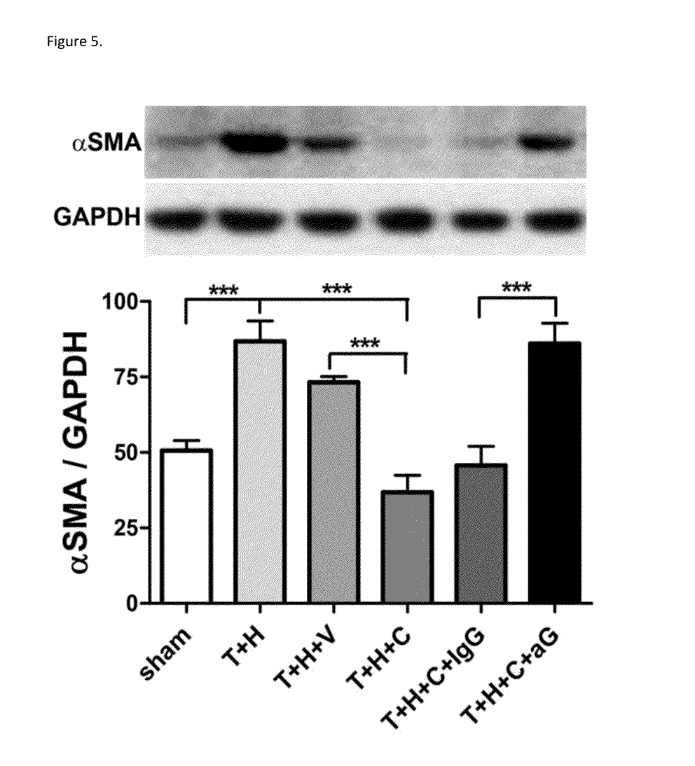

As shown in FIG. 5, Sham hippocampi expressed a very low level of the α-SMA protein, whereas TBI plus hemorrhagic shock significantly increased α-SMA expression on the injury side 15 days post-injury. On the other hand, transplantation of human neural stem cells (hNSCs) one day post injury prevented the increased expression of α-SMA in injured hippocampi, an effect blocked by simultaneous treatment with GDNF neutralizing antibody.

### Conclusion

In summary, the increased α-SMA expression at the sub-acute stage after TBI and a secondary insult could be reversed by human neural stem cell grafting. This protective effect is due to increased GDNF following human neural stem cell transplantation based on the data shown here and Gao et al., (Gao et al., 2006, Exp Neurol 201:281-92). Second, multiple brain injuries result in a broader change of α-SMA expression in both ipsilateral and contralateral hippocampal regions, which lasts for at least 2 weeks. Third, intrahippocampal infusion of the GDNF neutralizing antibody seems to have a profound effect on α-SMA expression, probably by blocking GDNF that is secreted from both grafted human neural stem cells.

### Example 8

### Targets for GDNF—Receptors and Signaling Pathways

GDNF elicits its neurotrophic effects through a multicomponent receptor complex including GDNF family receptor α1 (GFRα1) and the coactivator rearranged during transfection (RET) receptor tyrosine kinase (Saarma and Sariola, 1999, Microsc Res Tech 45 (4-5):292-302, Sariola and Saarma, 2003, J Cell Sci 116 (Pt 19):3855-3862). The assembling of this complex leads to autophosphorylation of RET on its tyrosine residues, which then stimulates the activation of downstream signal proteins including the phosphoinositide 3-kinase (PI3K)/Akt (or protein kinase B) and MAPK (extracellular-signal-related kinases or ERK1/2) pathways, which are important for neuronal survival (Airaksinen and Saarma, 2002, Nat Rev Neurosci 3 (5):383-394). GDNF also inhibits RhoA/ROCK signaling via activating ERK1/2 (Yoong et al., 2009, Mol Cell Neurosci 41 (4):464-473) and then promotes neurite outgrowth (Akerud et al., 1999, J Neurochem 73 (1):70-78, Coulpier and Ibanez, 2004, Mol Cell Neurosci 27 (2):132-139). Both receptors/co-activators are expressed in human and rat hippocampal tissues (Serra et al., 2005, Int J Dev Neurosci 23 (5):425-438). Furthermore, GFRα1 and RET expression in rat cortical neurons were increased 3 days after lateral fluid percussion injury (Bakshi et al., 2006, Eur J Neurosci 23 (8):2119-2134).

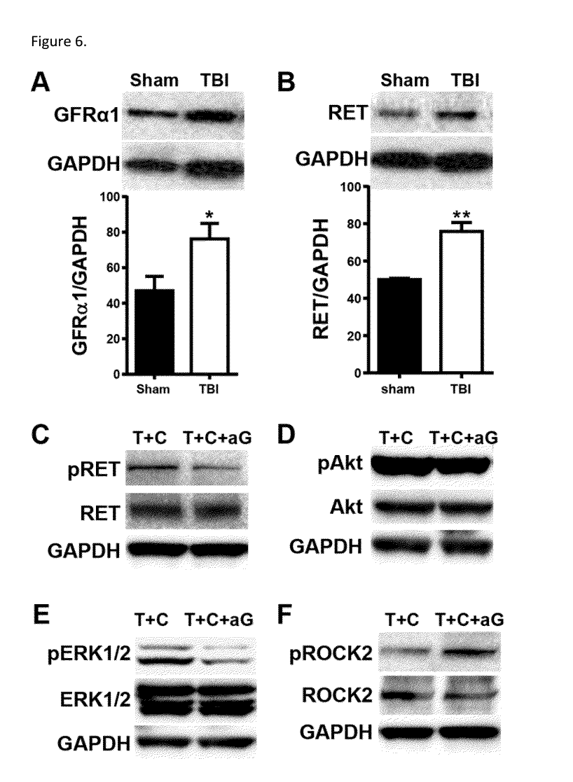

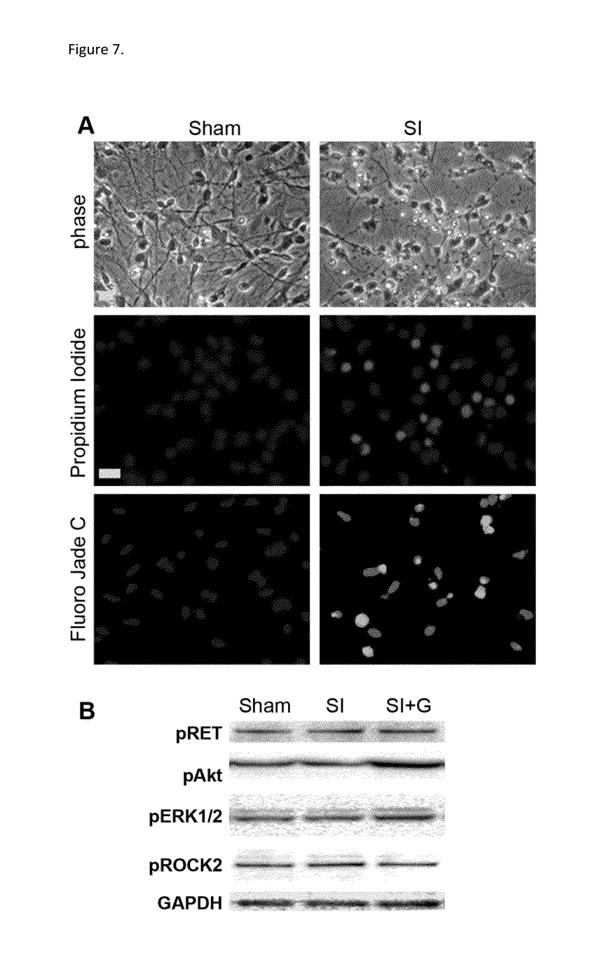

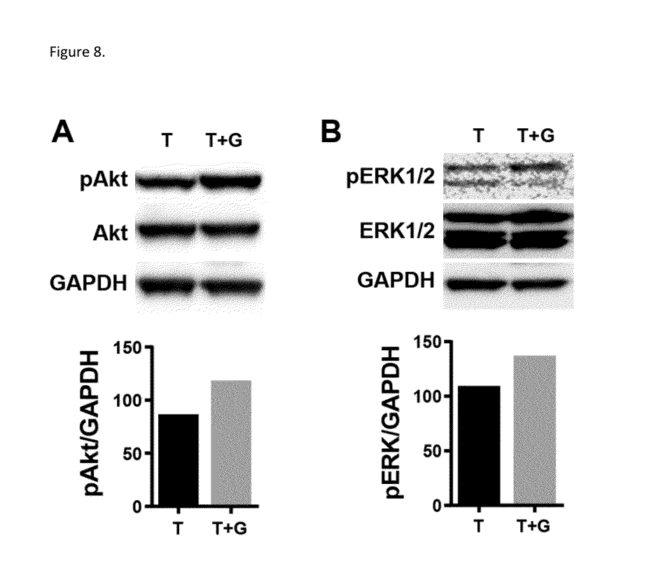

Western blot analyses were performed on protein extracts from adult male rat hippocampi 15 days post-injury. We detected increased expressions of GFRα1 and RET in rat hippocampi 2 weeks after TBI (FIG. 6). However, the downstream signaling changes of GDNF, particularly after TBI and GDNF treatments, remain unknown. To prove the feasibility of assessing GDNF targets, we conducted western blotting analyses on human neural stem cell-derived neurons/astrocytes following a moderate stretch injury as we described previously with minor modifications (Wang et al., 2012, J Neurotrauma 29 (2):295-312). Briefly, human neural stem cells were seeded to BioFlex plates and differentiated into neurons/astrocytes for 10 days, which were then subjected to 60 psi (regulator pressure) stretch injury under the Cell Injury Controller II. Thirty minutes later, cells were treated with 15 ng/ml GDNF or vehicle; and then subjected to morphological testing and Western blot analyses at 1.5 hrs post-injury. Stretch injury resulted in cell death determined by morphology, Propidium iodide and Fluoro Jace C staining (FIG. 7A). Little or no changes in signaling molecules were detected, whereas treatment with GDNF at 30 min post injury increased phosphorylated RET (pRET), pAkt and pERK1/2, but decreased pROCK2 at 1.5 hrs after injury (FIG. 7B). Besides the above in vitro stretch injury model, we also assessed the GDNF downstream signaling molecules by Western blot analyses in pig brains. As shown in FIG. 8, intranasal GDNF treatment 1 hr after TBI increased the phosphorylation of both Akt and ERK1/2 in the injured hippocampus at 30 min after treatment. Thus these data confirms that the intranasal administration of GDNF is efficient and effective.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. Supplementary materials referenced in publications (such as supplementary tables, supplementary figures, supplementary materials and methods, and/or supplementary experimental data) are likewise incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

What is claimed is:

1. A method for treating an acute axonal injury comprising intranasally administering to a subject in need thereof an effective amount of a composition comprising a compound having the biological activity of inhibiting the effect of rapid stretch injury on neural stem-cell derived neurons, and a pharmaceutically acceptable carrier.

2. The method of claim 1 wherein the compound comprises a Ret receptor ligand.

3. The method of claim 2 wherein the Ret receptor ligand comprises a GDNF polypeptide.

4. The method of claim 1 wherein the compound is not associated with a delivery reagent.

5. The method of claim 1 wherein the compound is associated with a delivery reagent.

6. The method of claim 5 wherein the delivery reagent comprises a liposome, micelle, polymersome, or nanparticle.

7. The method of claim 3 wherein the GDNF polypeptide is r-metHuGDNF.

8. The method of claim 1 wherein at least one dose is administered within 1 hour or within 6 hours after an acute axonal injury.

9. The method of claim 8 wherein the acute axonal injury is a diffuse axonal injury, a focal axonal injury, a mild acute axonal injury, a repetitive traumatic brain injury, or a spinal cord injury.

10. The method of claim 1 wherein the composition is formulated as an intranasal spray, an intranasal aerosol, or a nasal drop.

11. The method of claim 1 further comprising intranasally administering stem cells to the subject, wherein the stem cells are neuronal stem cells or adipose-derived stem cells.

12. The method of claim 11 wherein prior to the administering the adipose-derived stem cells are cultured in conditions suitable for differentiation of the adipose-derived stem cells into neuronal cells.

13. The method of claim 11 wherein the subject has a moderate or severe acute axonal injury.

14. A pharmaceutical formulation for intranasal administration comprising at least 1 ug/ul Ret receptor ligand, wherein the Ret receptor ligand is not associated with a delivery reagent.

15. A method for treating an acute axonal injury comprising intranasally administering to a subject in need thereof a composition comprising a Ret receptor ligand and a pharmaceutically acceptable carrier.

16. The method of claim 15 further comprising intranasally administering to the subject stem cells, wherein the stem cells are a neuronal stem cell or an adipose-derived stem cell.

17. The method of claim 16 wherein the adipose-derived stem cells are cultured in conditions suitable for differentiation of the adipose-derived stem cells into neuronal cells.

18. The method of claim 16 wherein the subject has a moderate or severe acute axonal injury.

19. A method for improving long term potentiation in a subject after an acute axonal injury comprising intranasally administering to a subject in need thereof a composition comprising a compound having the biological activity of inhibiting the effect of rapid stretch injury on neural stem-cell derived neurons and a pharmaceutically acceptable carrier, wherein the administration results in decreasing impairment of long term potentiation of hippocampal synapses.

20. The method of claim 19 wherein the compound comprises a Ret receptor ligand.

21. The method of claim 20 wherein the Ret receptor ligand comprises a GDNF polypeptide.

22. The method of claim 19 further comprising intranasally administering stem cells to the subject, wherein the stem cells are a neuronal stem cell or an adipose-derived stem cell.

23. The method of claim 22 wherein prior to the administering the adipose-derived stem cells are cultured in conditions suitable for differentiation of the adipose-derived stem cells into neuronal cells.

24. The method of claim 19 wherein the subject has a moderate or severe acute axonal injury.


SEQUENCE LISTING (1 sequence)

SEQ ID NO: 1 - protein, 135 aa, Homo sapiens
MSPDKQMAVLPRRERNRQAAAANPENSRGKGRRGQRGKNRGCVLTAIHLNVTDLGLGYETKEELIFRYCSGSCDAAETTYDKILKNLSRNRRLVSDKVGQACCRPIAFDDDLSFLDDNLVYHILRKHSAKRCGCI